(19) United States
(12) Patent Application Publication
(10) Pub. No.: US 2009/0105222 A1
(43) Pub. Date: Apr. 23, 2009

(54) PREVENTION AND TREATMENT OF FUNCTIONAL SOMATIC DISORDERS, INCLUDING STRESS-RELATED DISORDERS

(75) Inventors: Jay D. Kranzler, La Jolla, CA (US); Srinivas G. Rao, Encinitas, CA (US)

Correspondence Address: DARBY & DARBY P.C., P.O. BOX 770, Church Street Station, New York, NY, 10008-0770, US

(73) Assignee: Cypress Bioscience, Inc., San Diego, CA (US)

(21) Appl. No.: 12/233,790

(22) Filed: Sep. 19, 2008

Related U.S. Application Data
(63) Continuation of application No. 10/424,212, filed on Apr. 24, 2003, now abandoned.
(60) Provisional application No. 60/375,068, filed on Apr. 24, 2002.
(60) Provisional application No. 60/464,288, filed on Apr. 18, 2003.

(51) Int. Cl. A61K 31/55; A61K 31/165; A61K 31/195; A61K 31/428; A61K 31/197; A61K 31/433; A61K 31/4168; A61K 31/485

(52) U.S. Cl. 514/217; 514/620; 514/561; 514/367; 514/567; 514/362; 514/401; 514/282

(57) ABSTRACT

Methods for the prevention or treatment of stress-related disorders by administering a therapeutically effective amount of a dual serotonin/norepinephrine reuptake inhibitor to an individual under stress are described. A triple monoamine reuptake inhibitor for serotonin/noradrenaline/dopamine may also be administered to an individual at risk for a stress-related disorder. In a preferred embodiment the compound is milnacipran and is prophylactically administered at an effective amount to delay or prevent stress-related disorders in an individual at risk.

This application is a continuation of U.S. Ser. No. 10/424,212, fielded Apr. 24, 2003, which claims priority to U.S. Ser. No. 60/375,068 entitled “Methods of treating Functional Somatic Disorders” filed Apr. 24, 2002 by Jay D. Kranzler and Srinivas G. Rao and to U.S. Ser. No. 60/464,288 entitled “Prevention and Treatment of Stress-Related Disorders” filed Apr. 18, 2003, Jay D. Kranzler and Srinivas G. Rao.

### FIELD OF THE INVENTION

The present invention relates to a method of preventing or treating functional somatic disorders (FSD), including stress-related disorders (SRD). In one particular aspect, the present invention relates to methods of treating or preventing functional somatic disorders with dual serotonin norepinephrine reuptake inhibitors that also have NMDA antagonistic activity. In another aspect, the present invention relates to methods of treating FSD in a person having one or more symptoms of FSD by simultaneously treating at least one somatic symptom and one central nervous system (CNS) symptom of the FSD. In a preferred embodiment, the present invention relates to methods of preventing or treating SRD with dual serotonin/norepinephrine reuptake inhibitors.

### BACKGROUND OF THE INVENTION

Stress-related disorders (SRD) are the cause of seventy-five to ninety percent of office visits to physicians. Stress can affect the onset of, or susceptibility to disease. It can also affect the progression or course of disease even when there is another underlying pathophysiology of the disease. Recovery from an existing disease can also be delayed due to stress.

A stressor is an event or other factor that disrupts the body's stable balance of temperature, blood pressure, and other functions. Because humans have sophisticated brains and thought processes, anticipating a disruption can also be a stressor. The body responds to the stressor with the stress-response which changes the secretions of various hormones to reestablish stability. The stress response can be triggered by injury, hunger, heat, cold, or chemical exposure. The stress response is useful in cases of brief urgency because it increases energy and blood pressure while temporarily limiting less essential functions such as reproduction, growth and digestion. However, diseases can result if the stress response is chronically activated. Examples include depression, ulcers, fibromyalgia, chronic fatigue syndrome, irritable bowel syndrome, and other physiological dysfunction.

There are numerous physiological processes that are altered in response to stress. Among these are altered cortisol, corticotropin, catecholamine and serotonin levels. These levels return to baseline after an acute stressor is removed (McEwen N Eng J Med 1998 338(3):171-179). These biochemical markers of stress in turn lead to ill health and psychosocial disorders. Consequently, stress plays a major role in physical and mental health.

SRDs encompass a broad class of physical disturbances that occur as a result of stress in an individual's environment. For example, stress is a contributing factor to high blood pressure, heart disease, headaches, colitis, irritable bowel syndrome, temporo-mandibular joint disorder, cancer, peptic ulcers, insomnia, skin disorders and asthma. Stress can also aggravate other conditions such as multiple sclerosis, diabetes, herpes, mental illness, substance abuse and psychiatric disorders characterized by the presence of violent or aggressive tendencies. Particularly, stress contributes to functional somatic disorders, affective disorders and major depressive disorder. These include disorders such as chronic fatigue syndrome (CFS), fibromyalgia (FMS), Gulf War Syndrome, anxiety and post-traumatic stress disorder (PTSD).

One of the prevailing theories on the mechanism of SRDs centers around dysfunction in the hypothalamic-pituitary axis. There are several neuroendocrine abnormalities which have been identified in stress-related disorders such as chronic fatigue syndrome, fibromyalgia and depression. Most of these are consistent with a low central corticotropin-releasing hormone (CRH) levels which lead to changes in catecholamines and glucocorticoids in the periphery and a blunted stress response. In CPS patients, there is a blunting of the hypothalamic pituitary adrenal (HPA) axis, including low 24-hour free cortisol excretion, increased adrenocortical sensitivity to adrenocorticotropic hormone (ACTH), and attenuated ACTH response to CRR. These abnormalities are consistent with a tertiary (hypothalamic) adrenal insufficiency (Sternberg J Rheumatol 1993 20:418-421; Bearn et al Biol Psychiatry 1995 37:245-252). In FMS, hyporesponsiveness of the adrenal glands has been observed with decreases in cortisol and an exaggerated pituitary response to CRH suggesting a primary adrenal insufficiency. Similar abnormalities suggesting a blunting of the HPA axis have also been noted in many of the less common chronic fatigues states, such as dysthymia or seasonal affective disorder and may also be involved in less-understood disorders such as Gulf War Syndrome (Gold et al N Eng J Med 1988 319:348-353; Meaney et al Ann N Y Acad Sci 1993 697:70-85; Vanderpool et al J Clin Endocrinol Metabl 1991 72:1382-1387).

Stressors that disrupt normal exercise or sleep patterns would also contribute to this endocrine imbalance and results in further sleep and exercise disturbances. A positive feedback loop thereby develops wherein fatigue and lack of exercise causes further stress thus causing early stage SRDs and exacerbating existing diseases to more serious levels.

Many therapies address SRDs after they manifest and become a serious health problem. There is a need for effective prophylactic therapies to prevent the onset of this positive feedback loop and the resulting SRDs.

An exemplary SRD is Functional Somatic Disorder (FSD) which is “characterized more by symptom, suffering, and disability than by consistently demonstrable tissue abnormality” (Barsky et al Ann Intern Med 1999; 130:910-921). FSDs, by some estimates, affects as much as 20% of the population. Examples of Functional Somatic Disorders (FSD) include Migraine and Tension Headaches (MTH), Irritable Bowel Syndrome (IDS), Premenstrual Dysphoric Syndrome (PMDD), Temporomandibular Disorder (TMD), Multiple Chemical Sensitivities (MCS), and Interstitial Cystitis (IC).

Symptoms common to all of these FSDs, to varying degrees, include pain, fatigue, and cognitive and/or memory difficulties (Aaron et al Ann Intern Med 2001; 134:868-881), and all are associated with a higher prevalence of sleep disorders (Aaron et al Arch Intern Med 2000; 160:221-227) and psychiatric disturbances (Katon et al Ann Intern Med 2001; 134:917-925) than would be found in the general population. The pain symptomotology prevalent in the FSDs is thought to be due to a generalized heightened perception of somatic and/or visceral sensory stimuli.

A particular difficulty with FSD is the incomplete understanding of the disorder's etiology and the biological, environmental and other factors that impact it. Given the perception of the different manifestations of FSD as being unrelated and generally being treated by different medical disciplines, these different manifestations and indications have been treated with sometimes the same and sometimes different medications. Some of the common medications currently employed to treat various manifestations of FSD include analgesics, hypnotics, immune suppressants, various other prescribed medications, and an array of non-prescription medications. No single pharmacological agent or combination of agents has been shown to be effective in the treatment of the various manifestations of these disorders. Because of the lack of widespread recognition of FSD as a single disorder, there is a deficiency of effective treatment regimens for FSD and there is a need to develop effective treatments. Owing to their common symptomotology, the functional somatic disorders are thought to be related. However, they manifest different major symptoms.

Historically, antidepressants (AD) have played a prominent role in the treatment of many of the FSDs. In fact, the responsiveness of many FSDs, in part or in whole, to treatment with multiple classes of AD has been used to suggest a common etiology to the FSD as a form of “Affective Spectrum Disorder” where both the Syndrome itself and the accompanying psychopathology share common pathophysiologic features. However, whereas antidepressants of various classes have profound effects upon other Affective Spectrum Disorder, the efficacy of AD is limited in FSD, particularly for the selective serotonin reuptake inhibitor (SSRI) drug class. Moreover, the nature and specifics of any such proposed common etiologies have not been described, nor has any causal relationship between symptoms been proposed or even implied in the Affective Spectrum Disorder. These points in particular are discussed in the following publications: Gruber et al Psychiatric Clinics of N. America 1996; 19:351-369, Hudson and Pope, Amer J Psychiatry 1990; 147:552-564, and in Hudson et al., Journal of Rheumatology 1989; 16:15-22. Multivariate Models suggest that 1) many factors contribute to symptom development; 2) no single factor is necessary to the development of the disorder; and, 3) these factors interact in different combinations. For example, psychological factors such as stress or somatization, can clearly exacerbate the symptoms of FSD.

In yet other approaches for explaining the comorbidity of the FSD, testable hypotheses are implied, as these explanations “take sides” in the choice of biology versus psychology as the primary cause of other accompanying symptoms. These models can be divided between those that consider the physical manifestations of FSD as primary versus others that focus on the psychiatric disturbance as primary. However, the clinical predictions of these paradigms are not entirely consistent with the results that have been empirically observed in the clinic. For example, antidepressants have been demonstrated as effective in the mood component of the FSD in almost all cases; however, their efficacy on the pain component of the syndrome has been far less consistent. Also, statistical analysis has supported the independence of the various FSD, even when controlled for level of psychiatric distress. See, in particular, Clauw Med Hypotheses 1995; 44:369-378; Mayer Gut 2000; 47:861-869; Barsky 1999; op cit; Robbins et al J Nerv Mental Dis 1997; 185:606-615; and Whorwell et al Gut 1986; 27:37-40.

The problem with all of the proposed models is that they provide no direction for selection of treatment for the patient, nor do they provide any direction for new drug development, as no hypothesis to be tested is generated by these explanations. There still exists significant need for the development of effective therapies for treatment of patients afflicted with FSDs.

It is an object of the present invention to provide an effective therapy to treat individuals under acute stress exhibiting mild signs of stress before the signs are exacerbated into serious SRDs.

It is a further object of this invention to provide methods to identify and treat individuals predisposed to developing SRDs with a compound to prevent the manifestation of SRDs.

It is a further object of this invention to provide methods to treat individuals under acute stress with a pharmaceutical composition before SRDs manifest until a time where the stressor is relieved.

### SUMMARY OF THE INVENTION

Methods for the prevention or treatment of stress-related disorders such as functional somatic syndrome (FSD) and/or the symptoms associated therewith has been developed. The method generally involves simultaneously treating at least one somatic symptom and one central nervous system (CNS) symptom of the FSD. In a preferred embodiment, a therapeutically effective amount of a dual serotonin norepinephrine reuptake inhibitor (“DRI”) compound of a specific type, or a pharmaceutically acceptable salt thereof is administered. The most preferred DRI compounds are non-tricyclic SNRIs, wherein serotonin reuptake inhibition is greater than norepinephrine reuptake inhibition; and NSRIs, wherein norepinephrine reuptake inhibition is greater than serotonin reuptake inhibition. The most preferred compound is milnacipran or a bioequivalent or pharmaceutically acceptable salt thereof. Other preferred compounds are duloxetine and venlfaxine or a bioequivalent or pharmaceutically acceptable salt thereof. In yet another embodiment, a therapeutically effective amount of a non-tricyclic triple reuptake inhibitor (“TRI”) compound of a specific type, or a pharmaceutically acceptable salt thereof, is administered. The TRI compounds are characterized by their ability to block the reuptake (and, hence, increase central concentrations of) the three primary brain monoamines: serotonin, noradrenaline, and dopamine.

### BRIEF DESCRIPTION OF THE FIGURE

FIG. 1 illustrates the bidirectional relationships between factors involved in functional somatic syndromes.

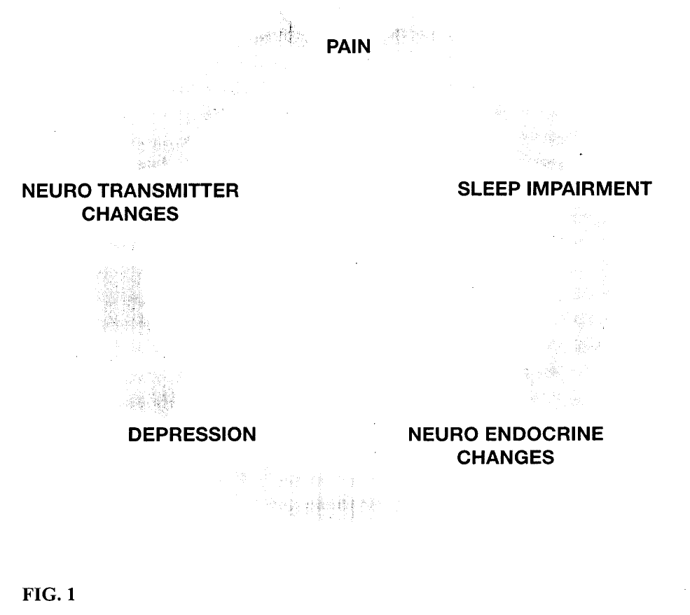

### DETAILED DESCRIPTION OF THE INVENTION

### Abbreviations

CFS chronic fatigue syndrome
FMS fibromyalgia syndrome
PTSD post-traumatic stress disorder
SRD stress-related disorder
FSD functional somatic disorder
5-HT serotonin
NE norepinephrine (noradrenaline)
NMDA N-methyl D-aspartate
NSAIDs non-steroidal anti-inflammatory drugs
SSRIs selective serotonin reuptake inhibitors
TCAs tricyclic antidepressants
SNRIs dual serotonin norepinephrine reuptake inhibitors. 5-HT>NE is implied
NSRI an alternative acronym for NE>5-HT SNRI
DA dopamine
TRI a compound that blocks the reuptake of 5-HT, NE, and DA
DRI a class of compounds that blocks the reuptake of 5-HT and NE. This class can be further broken into SNRI and NSRI subclasses.

### DEFINITIONS

The term “dual serotonin norepinephrine reuptake inhibitor compound” (also referred herein as DRI compounds) refers to the well-recognized class of anti-depressant compounds that inhibit reuptake of serotonin and norepinephrine. Common DRI compounds include, but are not limited to, venlafaxine, duloxetine, and milnacipran.

The term “NE>5-HT SNRI” or “NSRI” refers to a particular subclass of DRI compounds that inhibit the reuptake of norepinephrine more than they inhibit reuptake of serotonin; this subclass is useful in particular embodiments of the methods and kits of the present invention, as will be described in more detail herein.

The term SNRI refers to the particular DRI compounds that inhibit the reuptake of serotonin more than they inhibit reuptake of norepinephrine.

The term TRI refers to a class of compounds with antidepressant, anorectic, and anti-Parkinsonian properties that inhibit the reuptake of serotonin, noradrenaline, and dopamine.

The term Migraine and Tension Headaches refers to disorders which result in headaches. Migraine which is usually a unilaterally throbbing headache accompanied by some or all of the following-nausea, vomiting, photophobia (dislike of lights), phonophobia (dislike of noise). Attacks last on average 4-72 hours, are of moderate to severe intensity and are made worse by movement. Tension headaches are a nonspecific type headache, which is not vascular or migrainous, and is not related to organic disease. It is caused by tightening of the muscles in the back of the neck and scalp.

The term Atypical Facial Pain refers to a syndrome encompassing a wide group of facial pain problems including burning, aching or cramping, occurs on one side of the face, often in the region of the trigeminal nerve and can extend into the upper neck or back of the scalp with few if any periods of remission.

The term Non-Cardiac Chest Pain refers to chest pain not caused by the heart. The most common cause of non-cardiac chest pain arises from the esophagus including gastroesophageal reflux disease (GERD) and esophageal spasm.

The term Irritable Bowel Syndrome refers to a disorder that interferes with the normal functions of the large intestine (colon). It is characterized by a group of symptoms—crampy abdominal pain, bloating, constipation, and diarrhea. IBS causes a great deal of discomfort and distress. It does not permanently harm the intestines but can be disabling for some people.

The term Premenstrual Dysphoric Disorder refers to a debilitating set of symptoms associated with the part of a woman's cycle that precedes her menstrual period and is also a psychiatric term for a major mood disturbance. PMDD symptoms are so severe that a woman's day-to-day activities are completely disrupted.

The term Temporomandibular Disorder refers to not just one disorder, but a group of conditions, often painful, that affect the jaw joint (temporomandibular joint, or TMJ) and the muscles that control chewing. These disorders are classified into 3 groups: myofascial pain, degenerative joint disease and internal derangement of the joint.

The term Multiple Chemical Sensitivities refers to a disorder in which individuals report multiple distressing symptoms after exposure to household or environmental substances that are not toxic or allergenic to most people.

The term Interstitial Cystitis refers to one of the chronic pelvic pain disorders, and is a condition resulting in recurring discomfort or pain in the bladder and the surrounding pelvic region. Symptoms may include an urgent need to urinate (urgency), frequent need to urinate (frequency), or a combination of these symptoms. Pain may change in intensity as the bladder fills with urine or as it empties.

The term Chronic Lower Back Pain refers to pain in the lumbar region that persists for longer than six months, even though it may not be constant.

### I. Stress-Related Disorders

There are numerous disorders that are known to be either caused by or exacerbated by stress. These include addictive disorders such as substance abuse, anorexia, bulimia, obesity, smoking addiction, and weight addiction; anxiety disorders such as agoraphobia, anxiety disorder, obsessive compulsive disorder, panic attacks, performance anxiety, phobias, and post-traumatic stress disorder; autoimmune diseases such as allergies, arthritis, fibromyalgia, fibromytosis, lupus, multiple sclerosis, rheumatoid arthritis, Sjogren's syndrome, and vitiligo; cancer such as bone cancer, brain cancer, breast cancer, cervical cancer, colon cancer, Hodgkin's disease, leukemia, liver cancer, lung cancer, lymphoma, multiple myeloma, ovarian cancer, pancreatic cancer, and prostate cancer; cardiovascular disorders such as arrhythmia, arteriosclerosis, Burger's disease, essential hypertension, fibrillation, mitral valve prolapse, palpitations, peripheral vascular disease, Raynaud's disease, stroke, tachycardia, and Wolff-Parkinson-White Syndrome; and developmental disorders such as attention deficit disorder, concentration problems, conduct disorder, dyslexia, hyperkinesis, language and speech disorders, and learning disabilities.

The most relevant stress-related disorders to the present method of treatment include functional somatic disorders (FSDs), anxiety disorders, and major depressive disorder.

a. Functional Somatic Disorders

Functional Somatic Disorders (FSD) include, without limitation: Chronic Fatigue Syndrome (CFS), Fibromyalgia Syndrome (FMS), Migraine and Tension Headaches (MTH), Irritable Bowel Syndrome (IBS), Atypical Facial Pain (AFP), Premenstrual Dysphoric Syndrome (PMDD), Temporomandibular Disorder (TMD), Non-Cardiac Chest Pain (NCCP), Multiple Chemical Sensitivities (MCS), Interstitial Cystitis (IC), Chronic Pelvic Pain (CPP), and subsets of chronic Lower Back Pain (LBP) and are characterized more by symptom, suffering and disability rather than tissue abnormality. Symptoms common to FSDs, to varying degrees, include pain, fatigue, and cognitive and/or memory difficulties (Aaron et al Ann Intern Med 2001; 134:868-881), and all are associated with a higher prevalence of sleep disorders (Aaron et al Arch Intern Med 2000; 160:221-227) and psychiatric disturbances (Katon et al Ann Intern Med 2001; 134:917-925) than would be found in the general population. The pain symptomotology prevalent in the FSDs is thought to be due to a generalized heightened perception of somatic and/or visceral sensory stimuli. Patients with FSDs often display abnormalities in pain perception in the form of both allodynia (pain with innocuous stimulation) and hyperalgesia (increased sensitivity to painful stimuli).

It is estimated that approximately 20-40% of individuals with FSD have an identifiable current mood disorder such as depression or anxiety disorder at the time of diagnosis. The lifetime prevalence of depression has been reported as being as high as 70%. (Boissevain, and MCain, Pain. 1991; 191:227-38; Boissevain and MCain, Pain. 1991; 45:239-48; Hudson et al Am J Psychiatry 1985; 142:441-6).

A particular difficulty with FSD is the incomplete understanding of the disorder's etiology and the biological, environmental and other factors that impact it. Given the perception of the different manifestations of FSD as being unrelated and generally being treated by different medical disciplines, these different manifestations and indications have been treated with sometimes the same and sometimes different medications. Some of the common medications currently employed to treat various manifestations of FSD include, but are not limited to, analgesics, hypnotics, immune suppressants, various other prescribed medications, and an array of non-prescription medications.

One particular FSD is Gulf War syndrome named after veterans of the 1990-1991 Persian Gulf War. The etiology is not well understood but the syndrome is characterized by the presence of symptoms such as chronic fatigue, muscle and joint pain, headaches, skin rashes, concentration and memory problems, respiratory problems, sleep disturbances, gastrointestinal disturbances and depression. Two types of Gulf War Syndrome have been identified based on the presence of select symptoms. Syndrome 1 (Impaired cognition) is characterized by depression, and concentration difficulties. It is commonly found in Gulf War veterans who wore pesticide-containing flea collars. Syndrome 2 (Confusion-Ataxia) is the most sever form and is characterized by impaired thinking and reasoning, dizziness, balance and coordination deficits. It is commonly found in Gulf War veterans who claimed to be exposed to nerve gas. Data indicate that veterans with this type have the most extensive brain damage (Haley et al. Neuroradiology 2000 215:807-817).

Although a broad array of medications are used in FSD patients, no single pharmacological agent or combination of agents has been shown to be effective in the treatment of the various manifestations of these disorders. Because of the lack of widespread recognition of FSD as a single disorder there is a deficiency of effective treatment regimens for FSD and there is a need to develop effective treatments.

b. Anxiety Disorder

Anxiety disorders, as a group, are the most common mental illness in America. More than 19 million American adults are affected by these debilitating illnesses each year. Children and adolescents can also develop anxiety disorders. Anxiety disorders are serious medical illnesses that affect approximately 19 million American adults. These disorders fill people's lives with overwhelming anxiety and fear. Unlike the relatively mild, brief anxiety caused by a stressful event such as a business presentation or a first date, anxiety disorders are chronic, relentless, and can grow progressively worse if not treated. The five major types of anxiety disorders are identified as: Panic Disorder, Obsessive-Compulsive Disorder, Post-Traumatic Stress Disorder, Generalized Anxiety Disorder and Phobias (including Social Phobia, also called Social Anxiety Disorder). Each anxiety disorder has its own distinct features, but they are all bound together by the common theme of excessive, irrational fear and dread. It is common for an anxiety disorder to accompany depression, eating disorders, substance abuse, or another anxiety disorder. Anxiety disorders can also co-exist with illnesses such as cancer or heart disease. In such instances, the accompanying disorders will also need to be treated. Before beginning any treatment, however, it is important to have a thorough medical examination to rule out other possible causes of symptoms.
i) Panic Disorder is characterized by repeated episodes of intense fear that strike often and without warning. Physical symptoms include chest pain, heart palpitations, shortness of breath, dizziness, abdominal distress, feelings of unreality, and fear of dying.
ii) Obsessive-Compulsive Disorder is characterized by repeated, unwanted thoughts or compulsive behaviors that seem impossible to stop or control.
iii) Post-Traumatic Stress Disorder is characterized by persistent symptoms that occur after experiencing or witnessing a traumatic event such as rape or other criminal assault, war, child abuse, natural or human-caused disasters, or crashes. Nightmares, flashbacks, numbing of emotions, depression, and feeling angry, irritable or distracted and being easily startled are common. Family members of victims can also develop this disorder. Post-traumatic stress disorder (PTSD) is a debilitating condition that can develop following a terrifying event. The event that triggers PTSD may be something that threatened the person's life or the life of someone close to him or her or it could be something witnessed.

Whatever the source of the problem, some people with PTSD repeatedly relive the trauma in the form of nightmares and disturbing recollections during the day. They may also experience other sleep problems, feel detached or numb, or be easily startled. They may lose interest in things they used to enjoy and have trouble feeling affectionate. They may feel irritable, more aggressive than before, or even violent. Things that remind them of the trauma may be very distressing, which could lead them to avoid certain places or situations that bring back those memories. Anniversaries of the traumatic event are often very difficult.

PTSD affects about 5.2 million adult Americans. Women are more likely than men to develop PTSD. It can occur at any age, including childhood, and there is some evidence that susceptibility to PTSD may run in families. The disorder is often accompanied by depression, substance abuse, or one or more other anxiety disorders. In severe cases, the person may have trouble working or socializing.
iv) Generalized Anxiety Disorder is characterized by exaggerated worrisome thoughts and tension about everyday routine life events and activities, lasting at least six months. Almost always anticipating the worst even though there is little reason to expect it; accompanied by physical symptoms, such as fatigue, trembling, muscle tension, headache, or nausea.
v) Phobias are characterized into two major types of phobias, social phobia and specific phobia. People with social phobia have an overwhelming and disabling fear of scrutiny, embarrassment, or humiliation in social situations, which leads to avoidance of many potentially pleasurable and meaningful activities. People with specific phobia experience extreme, disabling, and irrational fear of something that poses little or no actual danger; the fear leads to avoidance of objects or situations and can cause people to limit their lives unnecessarily.

c. Major Depressive Disorder

Major depressive disorder refers to a class of syndromes characterized by negative affect and repeated episodes of depression without any history of independent episodes of mood elevation and over-activity that fulfill the criteria of mania. Multiple subtypes of major depressive disorders are recognized, including these with atypical characteristics, psychotic components, etc. The age of onset and the severity, duration and frequency of the episodes of depression are all highly variable. The average age on onset is the late 20s but the disorder may begin at any age. The symptoms of major depressive disorder typically develop over days to weeks. Prodromal symptoms include generalized anxiety, panic attacks, phobias or depressive symptoms and may occur during several months preceding the episode. Individual episodes also last between 3 and 12 months but recur less frequently. Recovery is usually complete between episodes but a minority of patients may develop a persistent depression mainly in old age. Individual episodes of any severity are often precipitated by stressful life events; in many cultures, both individual episodes and persistent depression are twice as common in women as in men. There is a genetic component involved with this illness being 1.5 to 3 times as common among those with a first-degree affected biological relative than the general population. Common symptoms of a depressive episode include reduced concentration and attention; reduced self-esteem and self-confidence; ideas of guilt and unworthiness, ideas or acts of self-harm or suicide; disturbed sleep; and diminished appetite. Frequently, a major depressive episode follows a psychosocial stressor, particularly death of a loved one, marital separation, childbirth or the end of an important relationship.

The lowered mood varies little from day to day and is often unresponsive to circumstances, yet may show a characteristic diurnal variation as the day goes on. As with manic episodes, the clinical presentation shows marked individual variations, and atypical presentations are particularly common in adolescence. In some cases, anxiety, distress, and motor agitation may be more prominent at times that the depression, and the mood change may also be masked by added features such as irritability, excessive consumption of alcohol, histrionic behavior, and exacerbation of pre-existing phobic or obsessional symptoms, or by hypochondria. For depressive episodes regardless of severity, a duration of at least two weeks is usually required for diagnosis, but shorter periods may be reasonable if symptoms are unusually severe and of rapid onset. The various subtypes respond differently to the various classes of antidepressants. For example, it has been demonstrated that patients with atypical depressive states respond best to monoamine oxidase inhibitors (MAO-I) rather than tricyclic antidepressants.

### II. Hypothalamic-Pituitary Axis Dysfunction

The hypothalamic-pituitary axis (HPA) has been implicated in the progression of SRDs (Clauw and Chrousos, Neuroimmunomod 1997 4:134-153) and serves as the link between a stressor, such as pain, and the individual's endocrine, autonomic, and behavioral response. Classically, the HPA is regarded as a system programmed to react to changes in the environment by producing chemical messengers that mediate physiological changes to maintain homeostasis (Chrousos (1998)Ann NY Acad Sci 851: 311-351). However, recent evidence complicates this simple model by suggesting that genetic influences, environmental factors early in life, and exposure to chronic stress can permanently affect the HPA, and predispose to the development of disease. While much of this work has been done in the context of understanding the contribution of these changes to the pathophysiology of affective disorders (Heim and Nemeroff 1999), similar mechanisms are believed to be operative in FSDs (Neeck and Crofford (2000) Rheum Dis Clin North Am 26(4): 989-1002.

The key mediator in the HPA cascade is corticotropin-releasing factor (CRH), a neuropeptide produced in the paraventricular hypothalamus in response to physical or psychological stress. CRH, in turn, stimulates the release of corticotropin (ACTH) from anterior pituitary cells, which prompts the secretion of glucocorticoids from the adrenal gland to elicit adaptive reactions to the perceived threat, such as increasing blood glucose levels (Neeck and Crofford (2000) Rheum Dis Clin North Am 26(4): 989-1002). CRH can also exert secondary inhibitory effects on growth hormone and thyroid-stimulating hormone (TSH) by functioning as a neurotransmitter, increasing the secretion of somatostatin from hypothalamic and cortical neurons (Peterfreund and Vale (1983) Endocrinology 112(4): 1275-8) and hypothalamic LHRH release (Frias, Puertas et al. (1997) Neurochem Res 22(2): 171-4). Simultaneous to activation of the HPA axis, an organism will react to stress with a “fight or flight” response, mediated by the autonomic nervous system and resulting in physiologic changes such as tachycardia and hypertension.

### III. Risk Factors

There are numerous risk factors that would predispose an individual to SRDs. These factors would identify candidate individuals for prophylactic treatment of stress-related disorders before the development of severe stress-related symptoms. Risk factors have been previously used to identify individuals predisposed to stress-related anxiety disorders such as PTSD and are also relevant. These include: (a) prior trauma, (b) prior psychological adjustment, (c) family history of psychopathology, (d) perceived life threat during the trauma, (e) posttrauma social support, (f) peritraumatic emotional responses, and (g) peritraumatic dissociation. Stressors perceived as inescapable or unavoidable or those accompanied by a lack of predictability or support, evoke the strongest adverse biological consequences.

Female gender is clearly a major risk factor and many stress-related disorders are more prevalent in females than males. Examples include CFS, FMS, PTSD, and major depressive disorder which are all more frequently manifested in females than males.

The environment in which the stressor is experienced is very important, and exposure to environments characterized by a loss of control, support, predictability are those associated with the highest likelihood of an acute stressor leading to a chronic illness. In this category fall situations such as childhood/developmental abuse. Studies have previously used previous trauma such as sexual trauma, general trauma, illicit drug use, pre-existing psychiatric disorders (most notably anxiety disorders and illicit drug use disorders). Early-life stressors can have a permanent impact on the subsequent biological response to stress in animals because of the plasticity of the nervous system. The plasticity may be due to changes in the numbers of neurons, number of circuits, and/or increases or decreases in gene expression, leading to permanent changes that define the function of the system. This may explain why individuals who develop FMS, CFS, somatoform disorders, IBS, and similar disorders display a higher than expected incidence of childhood physical and sexual abuse (Walling et al Obstet Gynecol 1994 84:200-206; Spaccarelli Psychol Bull 1994 116:340-362; Bendixen et al Child Abuse Negl 1994 18:837-847).

There are likely genetic risk factors that predispose an individual to having chronic sequalae of acute stressors. It is possible that the genetic predisposition to develop this spectrum of disorders is actually due to inherited differences in the activity of the stress response. Baseline abnormalities in the human stress response such as hyper-or hypo-activity in the hypothalamic-pituitary adrenal axis or autonomic nervous system may predispose to chronic SRD. Stressors that disrupt normal exercise or sleep patterns may put an individual at high risk of developing a chronic SRD.

Preclinical findings strongly implicate a role for CRH in the pathophysiology of certain anxiety disorders, probably through effects on central noradrenergic systems (Arborelius et al J Endocrinol 1999 160(1): 1-12). Noradrenaline has been implicated in patients with depression and affected by stress (Leonard J Psychiatry Neurosci 2001 26 Suppl:S11-6). There has been no previous report of using a mixed noradrenergic/serotonergic agent transiently after and acute stressor to prevent these sequalae. Such a compound would augment central noradrenergic and serotonergic systems, compensating for the low activity that predisposes individuals to these sequalae, until the acute pain, fatigue, distress resolves, and they are able to begin sleeping and exercising normally again.

### IV. Compositions

In a preferred embodiment a monoamine reuptake inhibitor is administered prophylactically to prevent the onset of SRDs. In a more preferred embodiment, an NSRI is administered after an acute stressor until the acute pain, fatigue and distress resolves and the individual can sleep and exercise normally again. In the most preferred embodiment, the NSRI is milnacipran.

This compound would preferably be administered in an effective amount to prevent the onset of one or more symptoms, or to alleviate the symptoms of stress-related disorders. The effective amount of compound to be administered would preferably prevent stress-related disorders from developing or being exacerbated into more serious conditions.

In one embodiment, TRI compounds, which inhibit the reuptake of serotonin, noradrenaline, and dopamine, are used to prevent or treat individuals with FSD or symptoms of FSD. Dopamine reuptake inhibitory activity typically involves blocking the dopamine transporter (DAT) such that dopamine reuptake is inhibited. The ability of a compound to block the DAT or increase release of dopamine can be determined using several techniques known in the art. For example, Gainetdinov et al., (1999, Science, 283: 397-401), describes a technique in which the extracellular dopamine concentration in the striatum can be measured using microdialysis. To determine the ability of a compound to block the DAT or increase the release of dopamine, the extracellular concentration of dopamine can be measured before and after administration of said compound. A statistically significant increase in dopamine levels post-administration of the compound being tested indicates that said compound inhibits the reuptake of dopamine or increases the release of dopamine. The ability to block the DAT can also be quantified with inhibitory concentration (IC) values, like IC₅₀, at the dopamine transporter. Several techniques for determining IC values are described in the art. (For example, see Rothman et al., 2000, Synapse, 35:222-227) The compounds useful in these methods typically have IC₅₀ values in the range of 0.1 nM to 600 μM. In particular, the compounds have IC₅₀ values of 0.1 nM to 100 μM.

A specific example of a TRI compound is sibutramine (BTS 54 524; N-[1-[1-(4-chlorophenyl)cyclobutyl]-3-methylbutyl]-N,N-dimethylamine hydrochloride monohydrate), or a pharmaceutically acceptable salt thereof. Sibutramine blocks the reuptake of the neurotransmitters dopamine, norepinephrine, and serotonin. The chemical structure of sibutramine is well known in the art. This compound is described in U.S. Pat. No. 4,939,175 and Buckett et al., (Prog. Nuero-Psychopharmacol. & Biol. Psychiat 1988 vol. 12:575-584).

Tricyclic antidepressants are a well-recognized class of antidepressant compounds and are characterized by a fused tricyclic nucleus. These are not preferred for use as described herein. Compounds that are commonly classified as tricyclic antidepressants include imipramine, desipramine, clomipramine, trimipramine, amitriptyline, nortriptyline, doxepin, and protriptyline.

In a preferred embodiment, the DRI compounds are NSRI compounds and exhibit a greater inhibition of norepinephrine reuptake than serotonin reuptake. In one embodiment, the NSRI compounds have a ratio of inhibition of norepinephrine reuptake to serotonin reuptake (“NE:5-HT”) of about 2-60:1. That is, the NSRI compound is about 2-60 times better at inhibiting reuptake of norepinephrine compared to inhibiting reuptake of serotonin. NE>5-HT SNRI compounds having a NE:5-HT ratio of about 10:1 to about 2:1 are thought to be particularly effective.

Various techniques are known in the art to determine the NE:5-HT of a particular SNRI. For example, the ratio can be calculated from IC₅₀ data for NE and 5-HT reuptake inhibition. It has been reported that for milnacipran the IC₅₀ of norepinephrine reuptake is 100 nM, whereas the IC₅₀ serotonin reuptake inhibition is 200 nM. See Moret et al., (Neuropharmacology, 24(12): 1211-1219, 1985); Palmier, C, et al. (1989). Therefore, the NE:5-HT reuptake inhibition ratio for milnacipran based on this data is 2:1. Of course, other IC values such as IC₂₅, IC₇₅, etc. could be used, so long as the same IC value is being compared for both norepinephrine and serotonin. The concentrations necessary to achieve the desired degree of inhibition (i.e., IC value) can be calculated using known techniques either in vivo or in vitro. See Sanchez and Hyttel (Cell Mol Neurobiol 19(4): 467-89); Turcotte et al (Neuropsychopharmacology. 2001 May; 24(5):511-21); Moret et al. (Neuropharmacology 1985 December; 24(12):1211-9.); Moret and Briley (Neuropharmacology. 1988 January; 27(1):43-9); Bel and Artigas (Neuropsychopharmacology 1999 December; 21(6):745-54); Palmier et al (Eur J Clin Pharmacol 1989; 37(3):235-8).

Examples of these NSRI compounds include milnacipran. Additional SNRI compounds that can be used include aminocyclopropane derivatives disclosed in WO95/22521; U.S. Pat. No. 5,621,142; Shuto et al. J. Med. Chem., 38:2964-2968, 1995; Shuto et al., J. Med. Chem., 39:4844-4852, 1996; Shuto et al., J. Med. Chem., 41:3507-3514, 1998; and Shuto et al., 85:207-213, 2001 that are structurally related to milnacipran and may thus inhibit the reuptake of norepinephrine more than they inhibit reuptake of serotonin can be used to practice the invention.

Milnacipran and methods for its synthesis are described in U.S. Pat. No. 4,478,836. Additional information regarding milnacipran may be found in the Merck Index, 12th Edition, at entry 6281. Unless specifically noted otherwise, the term “milnacipran” as used herein refers to both enantiomerically pure forms of milnacipran as well as to mixtures of milnacipran enantiomers.

Another specific example of an SNRI compound is duloxetine, or a pharmaceutically acceptable salt thereof. Duloxetine is usually administered to humans as the hydrochloride salt and most often administered as the (+) enantiomer. The chemical structure of duloxetine is well known to those skilled in the art. Duloxetine and methods for its synthesis are described in U.S. Pat. No. 4,956,388. Additional information regarding duloxetine may be found in the Merck Index, 12th Edition, at entry 3518.

Yet another specific example of an SNRI compound is venlafaxine, or a pharmaceutically acceptable salt thereof. The chemical structure of venlafaxine is well known to those skilled in the art. Venlafaxine and methods for its synthesis are described in U.S. Pat. Nos. 4,535,186 and 4,761,501. Additional information regarding venlafaxine may be found in the Merck Index, 12th Edition, at entry 10079. It is understood that venlafaxine as used herein refers to venlafaxine's free base, its pharmaceutically acceptable salts, its racemate and its individual enantiomers, and venlafaxine analogs, both as racemates and as their individual enantiomers.

Those of skill in the art will recognize that SNRI compounds such as milnacipran may exhibit the phenomena of tautomerism, conformational isomerism, geometric isomerism and/or optical isomerism. For example, as is clear from the above structural diagram, milnacipran is optically active. It has been reported in the literature that the dextrogyral enantiomer of milnacipran is about twice as active in inhibiting norepinephrine and serotonin reuptake than the racemic mixture, and that the levrogyral enantiomer is much less potent (see, e.g., Spencer and Wilde, 1998, supra; Viazzo et al., 1996, Tetrahedron Lett. 37(26):4519-4522; Deprez et al., 1998, Eur. J. Drug Metab. Pharmacokinet. 23(2): 166-171). Accordingly, milnacipran administered in enantiomerically pure form (e.g., the pure dextrogyral enantiomer) or as a mixture of dextrogyral and levrogyral enantiomers, such as a racemic mixture. Methods for separating and isolating the dextro-and levrogyral enantiomers of milnacipran and other SNRI compounds are well-known (see e.g., Grard et al., 2000, Electrophoresis 200021:3028-3034).

It will also be appreciated that in many instances the SNRI compounds may be metabolized to produce active SNRI compounds and that active metabolites could be used.

Glutaminergic neurotransmission plays a key role in the central sensitization that can cause the hypersensitivity sometimes associated with SRD. Therefore compounds that inhibit glutaminergic neurotransmission, like NMDA antagonists, can be particularly useful in treating SRD. It has been reported that milnacipran and its derivatives have antagonistic properties at the NMDA receptor. See Shuto et al., 1995, J. Med. Chem., 38:2964-2968; Shuto et al., 1996, J. Med. Chem., 39:4844-4852; Shuto et al., 1998, J. Med. Chem., 41:3507-3514; and Shuto et al., 2001, Jpn. J. Pharmacol., 85:207-213. The SNRI compounds with NMDA receptor antagonistic properties can have IC₅₀ values from about 1 nM-100 μM. For example, milnacipran has been reported to have an IC₅₀ value of about 6.3 μM. The NMDA receptor antagonistic properties of milnacipran and its derivatives are described in Shuto et al., 1995, J. Med. Chem., 38:2964-2968; Shuto et al., 1996, J. Med. Chem., 39:4844-4852; Shuto et al., 1998, J. Med. Chem., 41:3507-3514; and Shuto et al., 2001, Jpn. J. Pharmacol., 85:207-213. Methods for determining the antagonism and affinity for antagonism are disclosed in Shuto et al., 1995, J Med. Chem., 38:2964-2968; Shuto et al., 1996, J. Med. Chem., 39:4844-4852; Shuto et al., 1998, J. Med. Chem., 41:3507-3514; and Shuto et al., 2001, Jpn. J. Pharmacol., 85:207-213. Aminocyclopropane derivatives disclosed in WO95/22521; U.S. Pat. No. 5,621,142; Shuto et al., J. Med. Chem., 38:2964-2968, 1995; Shuto et al., J. Med. Chem., 39:4844-4852, 1996; Shuto et al., J. Med. Chem., 41:3507-3514, 1998; and Shuto et al., Jpn. J. Pharmacol., 85:207-213, 2001 that inhibit reuptake of NE more than 5-HT and have NMDA antagonistic properties can be.

The SNRI compounds, for example, milnacipran, can be administered adjunctively with other active compounds such as antidepressants, analgesics, muscle relaxants, anorectics, stimulants, antiepileptic drugs, and sedative/hypnotics. Specific examples of compounds that can be adjunctively administered with the SNRI compounds include, but are not limited to, neurontin, pregabalin, pramipexole, L-DOPA, amphetamine, tizanidine, clonidine, tramadol, morphine, tricyclic antidepressants, codeine, carbamazepine, sibutramine, amphetamine, valium, trazodone and combinations thereof. Typically, for an SRD patient, the SNRI compound may be adjunctively administered with antidepressants, anorectics, analgesics, antiepileptic drugs, muscle relaxants, and sedative/hypnotics. Adjunctive administration, as used herein, means simultaneous administration of the compounds, in the same dosage form, simultaneous administration in separate dosage forms, and separate administration of the compounds. For example, milnacipran can be simultaneously administered with valium, wherein both milnacipran and valium are formulated together in the same tablet. Alternatively, milnacipran could be simultaneously administered with valium, wherein both the milnacipran and valium are present in two separate tablets. In another alternative, milnacipran could be administered first followed by the administration of valium, or vice versa.

These compounds would preferably be administered in an effective amount to prevent the onset of one or more symptoms, or to alleviate the symptoms of stress-related disorders. The effective amount of compound to be administered would preferably prevent stress-related disorders from developing or being exacerbated into more serious conditions.

The SNRI compounds can be administered therapeutically to achieve a therapeutic benefit or prophylactically to achieve a prophylactic benefit. By therapeutic benefit is meant eradication or amelioration of the underlying disorder being treated, e.g., eradication or amelioration of the underlying SRD, and/or eradication or amelioration of one or more of the symptoms associated with the underlying disorder such that the patient reports an improvement in feeling or condition, notwithstanding that the patient may still be afflicted with the underlying disorder. For example, administration of milnacipran to a patient suffering from SRD provides therapeutic benefit not only when the underlying SRD is eradicated or ameliorated, but also when the patient reports decreased symptoms of any particular syndrome the SRD in the patient, for example, decreased fatigue, improvements in sleep patterns, and/or a decrease in the severity or duration of pain.

### V. Methods of Use

For therapeutic administration, the SNRI compound typically will be administered to a patient already diagnosed with the particular indication being treated.

For prophylactic administration, the SNRI compound may be administered to a patient at risk of developing SRD, or to a patient reporting one or more of the physiological symptoms of SRD, even though a diagnosis of SRD may not have yet been made. Alternatively, prophylactic administration may be applied to avoid the onset of the physiological symptoms of the underlying disorder, particularly if the symptom manifests cyclically. In this latter embodiment, the therapy is prophylactic with respect to the associated physiological symptoms instead of the underlying indication. For example, the SNRI compound could be prophylactically administered prior to bedtime to avoid the sleep disturbances associated with SRD. Alternatively, the SNRI compound could be administered prior to recurrence or onset of a particular symptom, for example, pain, or fatigue.

a. Individual Evaluation

An individual can be assessed based on risk factors described above and to determine whether or not a predisposition exists to develop SRD. Therapy can be administered if an individual is determined to be significantly at risk or has been acutely exposed to a stressor. In a preferred embodiment the compound will be administered prior to onset of any stress-related symptoms.

Psychophysiological Stress Tests can be performed to measure the amount of stress-induced anxiety present in the various systems of the body (i.e. muscular, cardiovascular, digestive, respiratory and neurological systems). These stress tests are routinely used in the art. Test results are compared to both local and national norms, to determine if the individual is exhibiting an excessive amount of physiological anxiety and whether or not they are able to recover from a standardized stressful stimuli in an appropriate length of time.

Psychological testing can be used to monitor those individuals belonging to the risk groups to determine the emotional and/or social etiology of the stress disorder. These tests are known in the art and include health-related assessments, mental health assessments, personality tests, and personality type assessment.

b. Formulation and Routes of Administration

The compounds, or pharmaceutically acceptable salts thereof, can be formulated as pharmaceutical compositions, including their polymorphic variations. Such compositions can be administered orally, buccally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. In the preferred embodiment the composition is administered orally.

Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980). The term “pharmaceutically acceptable salt” means those salts which retain the biological effectiveness and properties of the compounds used in the present invention, and which are not biologically or otherwise undesirable. Such salts may be prepared from inorganic and organic bases. Salts derived from inorganic bases include, but are not limited to, the sodium, potassium, lithium, ammonium, calcium, and magnesium salts. Salts derived from organic bases include, but are not limited to, salts of primary, secondary and tertiary amines, substituted amines including naturally-occurring substituted amines, and cyclic amines, including isopropylamine, trimethylamine, diethylamine, triethylamine, tripropylamine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, and N-ethylpiperidine. It should also be understood that other carboxylic acid derivatives, for example carboxylic acid amides, including carboxamides, lower alkyl carboxamides, di(lower alkyl) carboxamides, could be used.

The active DRI compounds (or pharmaceutically acceptable salts thereof) may be administered per se or in the form of a pharmaceutical composition wherein the active compound(s) is in admixture or mixture with one or more pharmaceutically acceptable carriers, excipients or diluents. Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

The compounds may be complexed with other agents as part of their being pharmaceutically formulated. The pharmaceutical compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); or lubricants. If any such formulated complex is water-soluble, then it may be formulated in an appropriate buffer, for example, phosphate buffered saline or other physiologically compatible solutions. Alternatively, if the resulting complex has poor solubility in aqueous solvents, then it may be formulated with a non-ionic surfactant such as Tween, or polyethylene glycol. Thus, the compounds and their physiologically acceptable solvates may be formulated for administration.

Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono-or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

The compounds may also be formulated in rectal compositions such as suppositories or retention enemas, e.g., containing conventional suppository bases such as cocoa butter or other glycerides. Suppositories for rectal or vaginal administration of the compounds discussed herein can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal or vaginal temperature, and which will therefore melt in the rectum or vagina and release the drug.

Solid dosage forms for oral administration may include capsules, tablets, pills, powders, and granules. In such solid dosage forms, the compounds of this invention are ordinarily combined with one or more adjuvants appropriate to the indicated route of administration. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

Alternatively, for oral administration, the pharmaceutical preparation may be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid) and sweetening, flavoring, and perfuming agents.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

The amount of active ingredient that can be combined with the carrier materials to produce a single dosage form will vary depending upon the patient and the particular mode of administration.

Preparations for oral administration may be suitably formulated to give controlled release of the active compound.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Dragee cores can be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.-All formulations for oral administration should be in dosages suitable for such administration.

For buccal administration, the compositions may take the form of tablets or lozenges formulated in conventional manner. The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active compound(s) may be in powder form for constitution with a suitable vehicle, e.g., sterile pyrogen-free water, before use.

In addition to the formulations described previously, the compounds may also be formulated as a depot or sustained-release preparation. Such long acting formulations may be administered by implantation, osmotic pump or transcutaneous delivery (for example subcutaneously or intramuscularly), intramuscular injection or a transdermal patch. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

c. Effective Dosages

Therapeutically effective amounts for use in humans can be determined from animal models. For example, a dose for humans can be formulated to achieve circulating concentration that has been found to be effective in animals. Useful animal models for these syndromes are known in the art. In particular, the following references provide suitable animal models of pain.

Effective amounts for use in humans can be also be determined from human data for the SNRI compounds used to treat depression. The amount administered can be the same amount administered to treat depression or can be an amount lower than the amount administered to treat depression. For example, the amount of milnacipran administered to prevent depression is in the range of about 50 mg-100 mg/day, or treat FSD, at more preferably 100 mg/day, and most preferably 200 mg/day for treatment.

Patient doses for oral administration of the SNRI compound typically range from about 1 μg-1 gm/day. For example, for the treatment of FSD, with milnacipran the dosage range is typically from 25 mg-400 mg/day, more typically from 100 mg-250 mg/day. The dosage may be administered once per day or several or multiple times per day. The amount of the SNRI compound will of course, be dependent on the subject being treated, the severity of the affliction, the manner of administration and the judgment of the prescribing physician.

1-26. (canceled)

27. A method for the treatment of anxiety disorders comprising administering to a patient in need thereof, an effective amount of milnacipran, or a pharmaceutically acceptable salt thereof.

28. The method of claim 27, wherein the anxiety disorder is panic disorder.

29. The method of claim 27, wherein the anxiety disorder is obsessive-compulsive disorder.

30. The method of claim 27, wherein the anxiety disorder is post-traumatic stress disorder.

31. The method of claim 27, wherein the anxiety disorder is generalized anxiety disorder.

32. The method of claim 27, wherein the anxiety disorder is a phobia.

33. A method for the treatment of anxiety disorders comprising administering to a patient in need thereof, an effective amount of a milnacipran, or a pharmaceutically acceptable salt thereof, and one or more agents selected from gabapentin, pregabalin, pramipexole, L-DOPA, amphetamine, tizanidine, clonidine, tramadol, morphine, a tricyclic antidepressant, codeine, carbamazepine, sibutramine, valium, and trazodone.

34. The method of claim 33, wherein the anxiety disorder is panic disorder.

35. The method of claim 33, wherein the anxiety disorder is obsessive-compulsive disorder.

36. The method of claim 33, wherein the anxiety disorder is post-traumatic stress disorder.

37. The method of claim 33, wherein the anxiety disorder is generalized anxiety disorder.

38. The method of claim 33, wherein the anxiety disorder is a phobia.

39. The method of claim 33, wherein the milnacipran, or a pharmaceutically acceptable salt thereof, is administered with gabapentin.

40. The method of claim 33, wherein the milnacipran, or a pharmaceutically acceptable salt thereof, is administered with pregabalin.

41. The method of claim 33, wherein the milnacipran, or a pharmaceutically acceptable salt thereof, is administered with pramipexole.
